(19) United States
(12) Patent Application Publication
(10) Pub. No.: US 2005/0182304 A1
(43) Pub. Date: Aug. 18, 2005

(54) Neuromonitoring system

(76) Inventors: James R. Petite JR., Woodstock, VT (US); Timothy J. Graettinger, Canonsburg, PA (US); Sean J. Maher, New Hope, PA (US)

Correspondence Address: SULLIVAN & WORCESTER LLP, ONE POST OFFICE SQUARE, BOSTON, MA, 02109, US

(21) Appl. No.: 11/037,956

(22) Filed: Jan. 18, 2005

Related U.S. Application Data
(60) Provisional application No. 60/536,724, filed on Jan. 16, 2004.
(60) Provisional application No. 60/544,888, filed on Feb. 14, 2004.

(51) Int. Cl. A61B 5/00

(52) U.S. Cl. 600/300

(57) ABSTRACT

Disclosed in one embodiment is a multimodal neuromonitoring system for providing clinicians with measures of important higher order, clinically relevant neurophysiological functions by combining the output of multiple medical devices each of which provides a measure of a more basic neurophysiological function. Also disclosed are electronic displays that present clinically meaningful relationships between various physical and chemical measurements of brain function, and a process for calculating when a patient's current neurophysiological state is different from a nominal “healthy” neurophysiological state.

### CROSS-REFERENCE TO RELATED APPLICATION

This application claims the benefit under 35 U.S.C. 119(e) of United States provisional applications entitled “Multimodal Neuromonitoring System,” U.S. Patent Application Ser. No. 60/536,724, filed on Jan. 16, 2004, and “Multimodal Neuromonitoring Alarm and Display System,” U.S. Patent Application Ser. No. 60/544,888, filed on Feb. 14, 2004; which are both herein incorporated by reference.

### FIELD OF THE INVENTION

This invention relates generally to neuromonitoring, with specific embodiments relating to multimodal neuromonitoring systems for providing clinicians with real-time, continuous measures of important higher order neurophysiological functions by combining the output of multiple neurosurgical monitoring devices, each of which provides a measure of a more basic neurophysiological function.

### BACKGROUND OF THE INVENTION

Routine neurosurgical monitoring has historically been largely limited to the measurement of intracranial pressure (ICP), a rather late stage indicator of the brain's status and probable clinical outcome. The past decade has seen the introduction of a number of sensor technologies which permit the monitoring of other important physical and chemical quantities including cerebral blood flow (CBF), partial pressures of tissue oxygen and carbon dioxide, pH, temperature, and the concentrations of various metabolites and ions via microdialysis. Cerebral perfusion pressure (CPP) has also been made available, and is calculated as the difference between mean arterial blood pressure and ICP.

### SUMMARY OF THE INVENTION

Systems according to the invention may be particularly advantageous in that they provide more meaningful data to clinicians by combining more basic measurements in more useful ways. While CPP, ICP and CBF are all important indicators in the clinical setting of traumatic brain injury, for example, when they are combined in a meaningful way to visualize and quantify cerebral autoregulation and vasoreactivity they can become much more powerful clinical assets.

Likewise, the partial pressure of oxygen in the brain tissue can be easily measured, but taken in isolation these data have not been nearly as useful clinically as when they are related to the metabolism of a region of brain tissue and meaningfully expressed, for example, as the cerebral metabolic rate of oxygen consumption (CMRO2).

And while prior art single-parameter devices may be suitable for the particular variable which they address, they do not algorithmically combine basic neurophysiological measurements to provide continuous, real-time measures of important higher level, clinically relevant neurophysiological functions such as cerebral autoregulation, CO2 reactivity, decoupling of CBF and partial pressure of tissue oxygen, and CMRO2. Further, conventional neurosurgical monitoring devices neither store data persistently in a database for future use nor display the aforementioned clinically relevant neurophysiological measures to clinicians in a real-time, continuous manner.

Systems according to the invention therefore substantially depart from the conventional concepts and designs of the prior art, and in so doing can provide clinicians with measures of important higher order, clinically relevant neurophysiological functions by combining the output of multiple medical devices each of which provides a measure of a more basic neurophysiological function.

More specifically, the invention features, in one general aspect, a patient neuromonitoring system that includes a capture device having a first input responsive to a first basic measure of patient function, and a second input responsive to a second basic measure of patient function for the same patient. A process is responsive to the capture device and operative to calculate at least one derived neurophysiological variable for the patient by combining the first and second basic measures of patient function, and to detect when the patient's state is different from a nominal state for the derived variable.

In preferred embodiments, the process can derive a measure of autoregulation from the first and second basic measures of patient function. The capture device can be responsive to cerebral blood flow measurements and arterial blood pressure measurements. The capture device can be responsive to cerebral perfusion pressure measurements and cerebral blood flow measurements. The process can derive a measure of carbon dioxide reactivity from the first and second basic measures of patient function. The process can derive a measure of carbon dioxide reactivity by comparing changes in the partial pressure of carbon dioxide and cerebral blood flow. The process can derive a measure of decoupling of cerebral blood flow and partial pressure of tissue oxygen from the first and second basic measures of patient function. The process can derive a measure of decoupling of cerebral blood flow by continually calculating measures indicating whether a relationship between cerebral blood flow increases and tissue oxygen increases is intact. The process can derive a measure of cerebral metabolic rate of oxygen consumption from the first and second basic measures of patient function. The process can derive a measure of cerebral metabolic rate of oxygen consumption from measures of cerebral blood flow, partial pressure of tissue oxygen, partial pressure of tissue carbon dioxide, intracranial pressure, and arterial blood pressure. The system can further include means for providing the at least one derived variable to a clinician. The means for providing the derived variables can include a display operative to simultaneously display a plurality of the derived variable on a graph. The means for providing the derived variables can include a display operative to display the at least one derived variable on a separate graph. The system can further include a database responsive to the capture device, wherein the capture device is operative to persistently store data from the first and second inputs. The capture device can further be operative to transform the output of various neurosurgical monitoring devices into a form that is suitable for storing in a database. The first and second inputs of the capture device can be responsive to the output of one or more neurosurgical monitoring devices. The capture device can be operative to receive analog or digital signals. The process can be operative to detect anomalous conditions from a plurality of rules. The process can further include a rule definition interface to define the rules. The capture device and the process can be operative on a continuous basis to obtain continuous values for the derived variable. The capture device and the process can be operative on a real-time basis to obtain real-time values for the derived variable. The nominal state can be derived from measurements made during a baseline period. The nominal state can be derived from a clinical understanding of the derived variable.

In another general aspect, the invention features a patient neuromonitoring method that includes receiving at least two different measures of patient condition from a patient, calculating at least one derived neurophysiological variable for the patient based on the two different measures of patient condition in a continuous manner, and detecting when the patient's state is different from a nominal state for the derived variable.

In preferred embodiments, the method can further include the step of outputting the derived neurophysiological variable to a clinician. The method can further include the step of displaying the derived neurophysiological variable to a clinician. The step of detecting can be responsive to a set of rules. The steps of receiving, calculating, and detecting can operate on a continuous basis. The steps of receiving, calculating, and detecting can operate on a real-time basis.

In a further general aspect, the invention features a patient neuromonitoring system that includes means for receiving at least two different measures of patient condition from a patient, means for calculating at least one derived neurophysiological variable for the patient based on the two different measures of patient condition in a continuous manner, and means for detecting when the patient's state is different from a nominal state for the derived variable.

In another general aspect, the invention features a patient neuromonitoring system that includes a capture device having a first input responsive to a first basic measure of patient function, and a second input responsive to a second basic measure of patient function for the same patient, and being operative to persistently store data from the first and second inputs, and a process responsive to the capture device and operative to calculate at least one derived neurophysiological variable for the patient in a continuous manner by combining the first and second basic measures of patient function.

In preferred embodiments, the system can further include a database responsive to the capture device, wherein the capture device is operative to persistently store data from the first and second inputs in the database. The system can further include a database responsive to the process, with the process being operative to persistently store values for the derived neurophysiological variable in the database.

In a further general aspect, the invention features a patient neuromonitoring method that includes receiving at least two different measures of patient condition from a patient, calculating at least one derived neurophysiological variable for the patient based on the two different measures of patient condition in a continuous manner, and persistently storing at least one of the two different measures of patient condition and the derived neurophysiological variable.

In another general aspect, the invention features a patient neuromonitoring system that includes a capture device having a first input responsive to a first basic measure of patient function, a second input responsive to a second basic measure of patient function for the same patient, and a third input responsive to a third measure of patient function for the same patient, and a process responsive to the capture device and operative to calculate at least one derived neurophysiological variable for the patient by combining a subset of the first, second, and third basic measures of patient function.

In preferred embodiments, the process can be responsive to the capture device and operative to calculate a plurality of different derived neurophysiological variables for the patient by combining subsets of the basic measures of patient function.

In a further general aspect, the invention features a patient neuromonitoring method that includes receiving different measures of patient condition from a patient, calculating a first derived neurophysiological variable for the patient based on a first subset of the different measures of patient condition in a continuous manner, and calculating a second derived neurophysiological variable for the patient based on a second subset of the different measures of patient condition in a continuous manner, wherein the first and second subsets are different.

It is an important object of this invention to provide an improved neuromonitoring system that combines data to provide more clinically relevant indicators of higher level physiological functions and patient health and thereby enable a clinician to better treat patients. Other objects and advantages of this invention will become obvious to the reader and it is intended that these objects and advantages be within the scope of this invention.

This invention may be embodied in the form illustrated in the accompanying drawings, attention being called to the fact, however, that the drawings are illustrative only, and that changes may be made in the specific construction illustrated.

### BRIEF DESCRIPTION OF THE DRAWINGS

FIG.1 is a schematic of a multimodal neuromonitoring system according to the invention;

FIG. 2 is a time plot of CBF and pBTO2;

FIG. 3 is a parametric plot of CBF versus pBTO2 that displays the nominal relationship and upper and lower confidence limits around the nominal relationship for the system of FIG. 1;

FIG. 4 is a set of diagrams showing an explicitly defined nominal relationship between CBF and pBTO2 for the system of FIG. 1;

FIG. 5 is a screen shot of a multi-variable display view for an embodiment of the neuromonitoring system of FIG. 1;

FIG. 6 is a screen shot of an autoregulation display view for an embodiment of the neuromonitoring system of FIG. 1;

FIG. 7 is a screen shot of a vasoreactivity display view for an embodiment of the neuromonitoring system of FIG. 1;

FIG. 8 is a screen shot of a metabolic index (O2/CBF) display view for an embodiment of the neuromonitoring system of FIG. 1;

FIG. 9 is a screen shot of a metabolic index (O2/CPP) display view for an embodiment of the neuromonitoring system of FIG. 1;

FIG. 10 is a screen shot of a multimodal display view for an embodiment of the neuromonitoring system of FIG. 1 showing an auxiliary control panel dialog;

FIG. 11 is a screen shot of an acquisition configuration dialog for the neuromonitoring system of FIG. 1;

FIG. 12 is a screen shot of a parameter range dialog for the neuromonitoring system of FIG. 1;

FIG. 13 is an application startup flowchart for the neuromonitoring system of FIG. 1;

FIG. 14 is a data acquisition timer flowchart for the neuromonitoring system of FIG. 1;

FIG. 15 is a timer update flowchart for the neuromonitoring system of FIG. 1;

FIG. 16 is a nominal curve calibration flowchart for the neuromonitoring system of FIG. 1; and

FIG. 17 is a rule definition dialog for use with the system of FIG. 1.

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### DESCRIPTION OF AN ILLUSTRATIVE EMBODIMENT

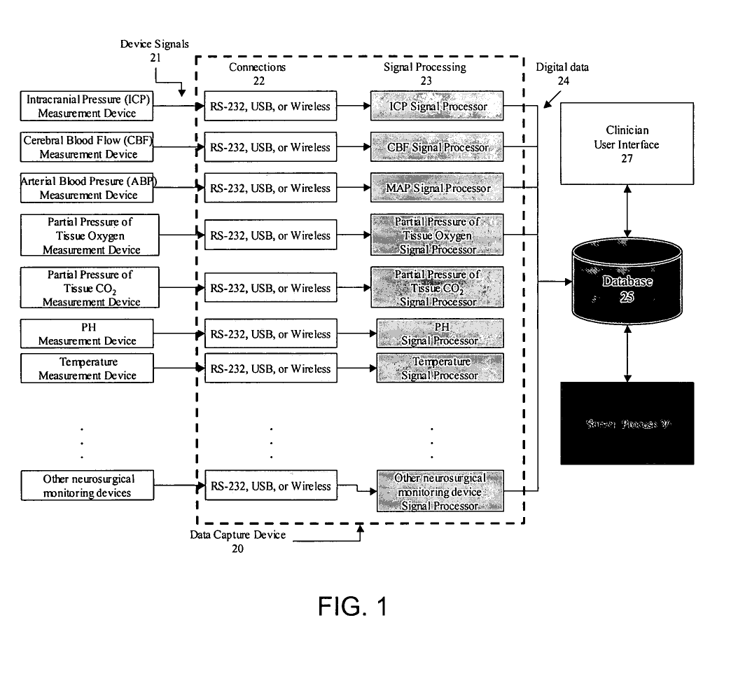

Referring to FIGS. 1-4, an illustrative multimodal neuromonitoring system according to the invention can interrelate measurements of various physical parameters and output derived quantities which are clinically relevant and thus useful to clinicians. This system can also include electronic displays that present clinically meaningful relationships between various physical and chemical measurements of brain function and a process for calculating when a patient's current neurophysiological state is different from a nominal “healthy” neurophysiological state. There are four major components of a process to detect when a patient's neurophysiological state is different from a nominal “healthy” neurophysiological state:
the measured condition of the patient
the definition of the nominal relationship between two or more physical or chemical measurements available through neurosurgical monitoring devices
the definition of deviation from the nominal relationship
the rules for detecting anomalies (unacceptable deviations from the nominal relationship)

These components will be described in detail below.

In the course of describing this invention, this application will refer to the following physical and chemical measurements of patient brain and neural health:
CBF—cerebral blood flow
CSF—cerebrospinal fluid
SAP—arterial pressure
SJO2—downstream venous oxygen partial pressure via jugular bulb
ICP—intracranial pressure
pBTO2—tissue oxygen partial pressure
eTCO2—end tidal carbon dioxide partial pressure
pBTCO2—tissue carbon dioxide partial pressure
Arterial O2—arterial oxygen partial pressure or pAO2

Other and improved measures of patient brain and neural health will become available in the future. This application anticipates these changes and improvements in measurements and monitors, and although this application calls out the particular measures above, this invention is not limited to those mentioned. Any measures of patient brain and neural health should be useable in connection with this invention.

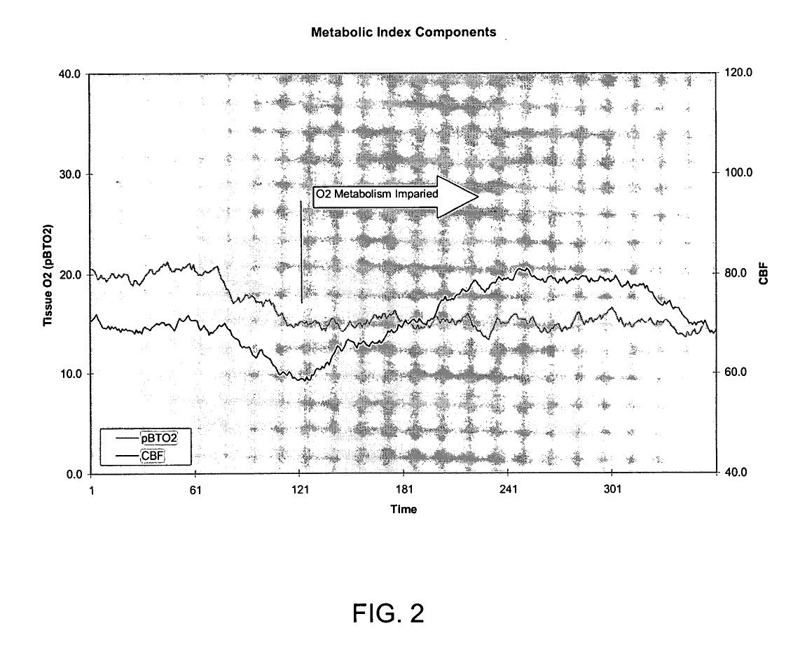

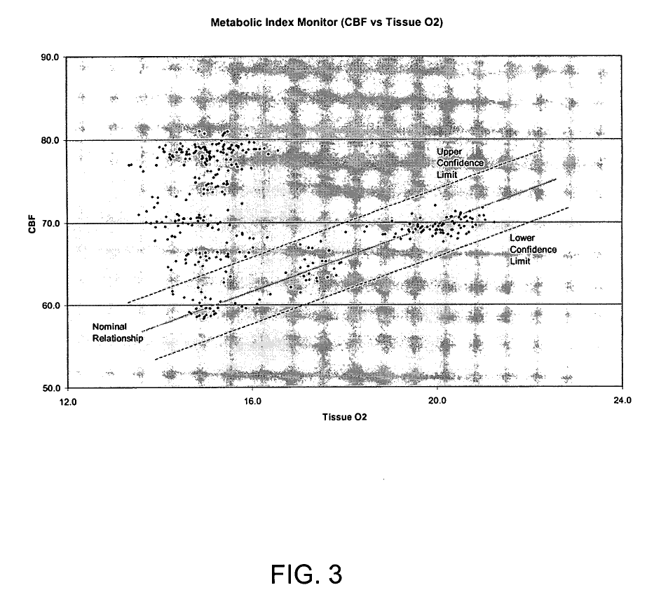

This application also anticipates that clinical personnel will interact with the present embodiment of the invention through a series of electronic displays. These displays can present clinically meaningful information in non-trivial, understandable ways. Displays associated with the preferred embodiment include, but are not limited to, the following:
Time plots: Time traces of multiple brain measurements, such as of pBTO2 and CBF, versus time. A sample is shown in FIG. 2.
Parametric plots: Parametric diagrams of multiple brain measurements versus each other, such as of CBF versus pBTO2, where each display point marks the paired quantities sampled at sequential points in time. A sample is shown in FIG. 3.
Event/Status/Alarm log: A text area of the display, where clinically important events are logged with time stamps and values of the relevant patient measurements. Indicators (e.g., colored green, yellow, and red text or symbols) indicate the patient status (nominal, transitional, exceptional, etc.).

Display technology is constantly evolving. Myriad configurations of the displays described above are possible, simultaneously in terms of dimensionality (number of measurements that are displayed in one chart, plot, or text area), number (number of display plots or elements), and devices (flat panels, virtual displays/goggles, etc). This application anticipates these changes in technology and configuration, among others, and their use should therefore fall within the scope of this invention.

The content of the displays can be critical to embodiments of this invention, but it is anticipated that the content of these displays will change as more is learned about the physical, chemical, and biological bases for the interactions occurring between the measured quantities. Changes in content that flow from this learning process should therefore fall within the scope of this invention. Based on the best current knowledge, the following content elements are defined as part of this illustrative embodiment:
Metabolic Index: This element tracks and displays the measured relationship between CBF and PBTO2 relative to the nominal relationship between these same quantities. Methods employed to define the nominal relationship and track its changes for the purpose of delivering alerts to clinical personnel are described in more detail below.
Autoregulation: This element tracks and displays the CBF and SAP relationship relative to the nominal relationship. As will be described in detail later, although the autoregulation display shows the CBF and SAP relationship relative to the nominal relationship, the definition of the nominal relationship and measures of deviation thereof include other measurements such as eTCO2, pBTCO2 and arterial O2.
Vasoreactivity: This element tracks and displays the CBF and pBTCO2 or eTCO2 relationship relative to the nominal relationship.
All Others: Other multidimensional combinations of the patient measurements listed above (CBF, pBTO2, etc.) can also produce meaningful clinical indicators of patient health. Though the descriptions below are limited to the indicators above (Metabolic Index, Autoregulation, etc.), this invention is applicable to any combination of patient brain and neural measurements.

One important aspect in delivering clinically relevant information about a patient's health to medical personnel is the present embodiment's ability to detect anomalies, or deviations from a nominal condition. For purposes of exposition, the application will focus on the workings of the invention in the context of the Metabolic Index. The methodology and workings of the invention are similar for the other indicators specified above.

There are four major components of the present embodiment to detect and display anomalies in the Metabolic Index for a patient. They are:
the measured condition of the patient
the definition of the nominal relationship between CBF and PBTO2 available through separate monitoring devices
the definition of deviation from the nominal relationship
the rules for detecting anomalies (unacceptable deviations from the nominal relationship).

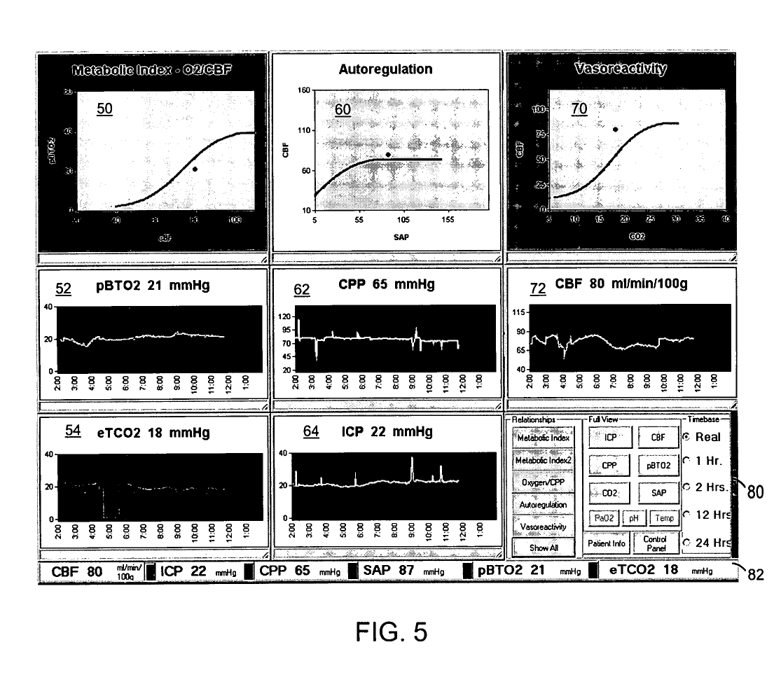

Each of these components are described below, along with a description of their interactions in the preferred embodiment of the invention. The dimensionality of the displayed relationship and dimensionality of the nominal relationship and calculations of deviation from that nominal relationship may be different. The displays are designed to help clinicians visually track important relationships between the physical and chemical measurements of patient brain and neural health and as such are influenced by clinician preference and training. The nominal relationship and calculations of deviation from that nominal relationship may use measurements that are not subsequently displayed. In FIG. 5, for example, the solid blue line shows the well-known nominal relationship between CBF and SAP that should hold if pressure autoregulation is working. This relationship and graph is well-known and easily understood by clinicians skilled in the art. The solid black and red lines in FIG. 5 show patient data plotted concurrently on the same graph, and indicates an alert that autoregulation may no longer be working. The underlying methodologies or algorithms that define the nominal relationship and calculations of deviation from that nominal relationship (which will be described in detail later) that trigger the alert shown in FIG. 5 may use arterial O2 and eTCO2 measurements in addition to CBF and SAP. Thus, while the display is two-dimensional showing CBF vs. SAP, the underlying methodology or algorithm may be four-dimensional. This difference in display and method or algorithm dimensionality may hold for any relationship.

The Measured Condition of the Patient: The CBF and pBTO2 of the patient are measured using standard instrumentation available in a neuro-intensive care unit (NICU) or similar facility. The data from these instruments are conditioned, time-stamped, and stored in the database 25 as shown in FIG. 1. From the database, the measured values are extracted as needed. This invention is not tied to any particular instrumentation technology, signal processing technology, computing technology, or data storage technology. Currently available products (e.g., a Neurosensor machine for CBF measurement and a Licox machine for pBTO2 measurement) provide the necessary capabilities for the present embodiment. This invention does, however, anticipate future improvements and changes to these and other underlying technologies, and these new technologies should therefore be useable in connection with future embodiments.

Nominal Relationship: The nominal relationship means the normal or standard interaction between two or more measured quantities. For the Metabolic Index, the quantities that define the nominal relationship are CBF and pBTO2. Experience indicates that the nominal relationship can be expressed and implemented in numerous ways. The present embodiment can include, but is not limited to, the following:
Patient Baseline: For a period of time, such as several hours to several days, patient data are collected and are designated as nominal. This means that, in a clinician's judgment, the relationship between CBF and pBTO2 is nominal. The data captured during this period are stored for later use to define the nominal relationship, as will be described below.
Peer Baseline: If a baseline is not available or not applicable for the current patient, baseline data from-one or more similar (peer) patients can be used to define the nominal relationship. The method for doing so will be described below.
Clinical Understanding: Clinical studies, empirical data, theoretical models, or the like can be used to define the nominal relationship, as will be detailed below.

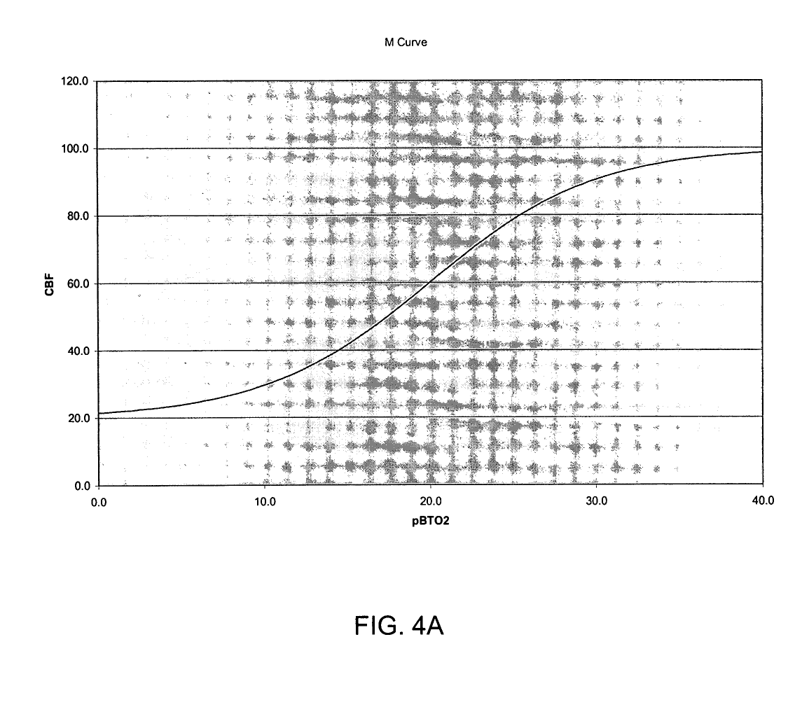

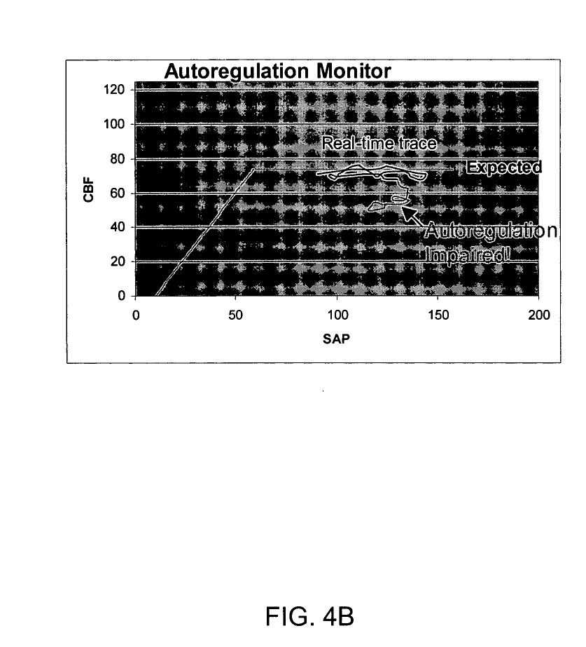

Based on a patient baseline, a peer baseline, or clinical understanding, the nominal relationship between CBF and pBTO2 is encapsulated in the present embodiment in two primary ways:
Implicitly: Using baseline data (patient and/or peer), the nominal relationship is simply defined by the “cloud” of data together with all of its statistical and mathematical properties.
Explicitly: Using a variety of data fitting and/or filtering techniques, (including but not limited to splines, piecewise-linear approximations, nonlinear regression, neural networks, and time delays, moving averages, low-pass filters, high-pass filters, and integrators), the nominal relationship can be expressed as an explicit mathematical formula. The choice of data fitting or filtering method, as well as the particular parameters employed therein, can be largely empirical. This application anticipates that new data fitting and filtering methods will become available, and these future methods should also be useable in connection with this invention. Further, the full range of fitting or filtering parameters for any of these methods should be usable in connection with this invention. In FIG. 4, the system displays a sample of an explicitly-defined nominal relationship between CBF and pBTO2.

Given any explicit theoretical model of neural metabolism, the nominal relationship between CBF and pBTO2 (and/or related brain and neural measurements) can be extracted as an explicit mathematical formula.

Deviation from the Nominal Relationship: Several methods can be used with a range of parameters to define deviation from the nominal relationship. Those methods used in the present embodiment of the invention are described below. The use of similar or other methods, the full range of parameter values for those methods, and tuning methods (manual or automatic/adaptive, online or off-line, or any combination thereof) can also be used in connection with the invention.

When the nominal relationship is defined implicitly via a collection of patient and/or peer data, deviation can be computed in several complementary ways:
Nearest Neighbor Distance: The distance between all neighboring data points in the data collection are computed. Various statistics about these distances are computed over the entire data collection. In the preferred embodiment, the neighborhood of a data point is defined as its five closest neighbors. Distance is defined as both the Euclidean distance and the Manhattan (right-angle) distance between data points. The deviation statistics computed in the preferred embodiment are: mean (average) distance, median distance, distance, maximum distance, standard deviation of distance, and mean absolute deviation of distance.
Convex polygon: From the collection of patient and/or peer data, one or more minimal convex polygons that “cover” the data are computed. Deviation is defined as data being outside any of the covering polygons.
Database match: The collection of patient and/or peer data can be encapsulated in a database, as mentioned above. Deviation can then be defined as newly measured patient data that does not match with any of the existing data in the database.

When the nominal relationship is defined explicitly via a mathematical formula, deviation can be computed in several complementary ways:
Confidence limits: When the explicit relationship was determined from patient and/or peer data, confidence limits on the relationship are developed either analytically (using well-known formulas) or empirically (using the data itself). Deviation is defined as data being outside the confidence limits, which themselves may be parameterized as 90%, 95%, 99% or like confidence limits. FIG. 3 illustrates the use of confidence limits to define deviation from the nominal relationship.
Experience: Based on clinical studies and experience, the acceptable ranges on the deviation from the nominal relationship can be defined clinically.

Rules for Detecting Anomalies: An anomaly refers to a patient condition that does not conform to the nominal relationship. A patient condition can be designated as an anomalous event based on the extent and duration of the non-conformance. Rules are defined below for use in present embodiment of the invention, but other clinically meaningful rules or combinations of rules based on the component elements described herein (measured condition, nominal relationship, deviation) could also be used. Further applicable to the invention are any combinations of fitting or filtering methods, the full range of parameter values for those methods, and any tuning techniques that are applicable to define the rules.

When the nominal relationship is defined implicitly, an anomalous event can be detected when:
the nearest neighbor distance of the measured patient condition is greater than the median distance for a period of 30 or more consecutive time samples
   OR
the 11-sample moving average of the measured patient condition is greater than the median distance
   OR
the measured patient condition is outside the collection of convex polygons for a period of 30 or more consecutive time samples.
   When the nominal relationship is defined explicitly, an anomalous event is detected when:
the measured patient condition is outside the 95% confidence limits for a period of at least 30 consecutive time samples. In FIG. 3, the patient measurements are outside the defined confidence limits for more than 30 samples and an anomalous event is detected
   OR
the 31-sample moving average of the measured patient condition is outside the 95% confidence limits
   OR
the measured patient condition is outside the experiential thresholds for a period of at least 30 consecutive time samples.

Existence of any or all of the conditions above triggers an event alarm that is time-stamped and logged in the database and on the display.

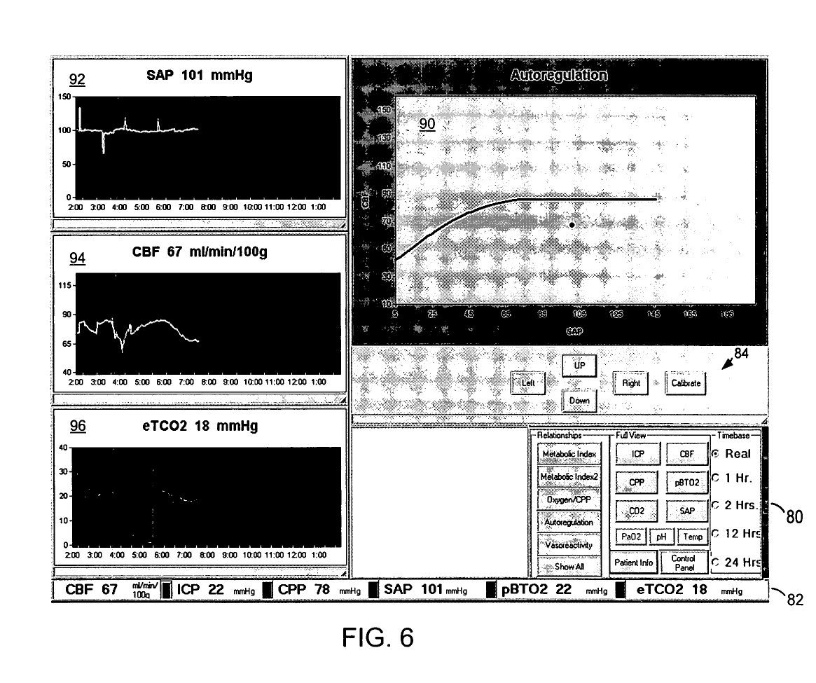

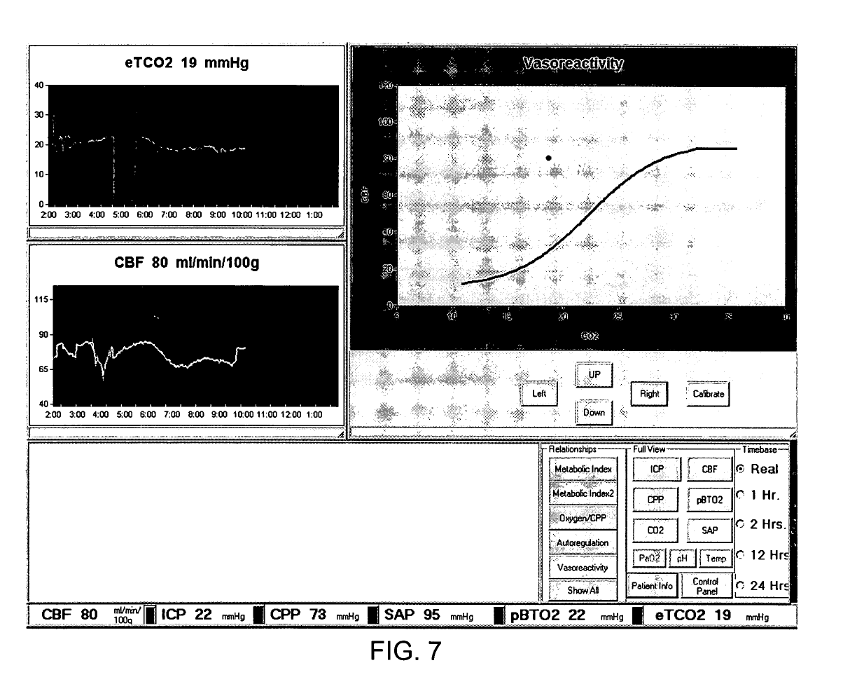

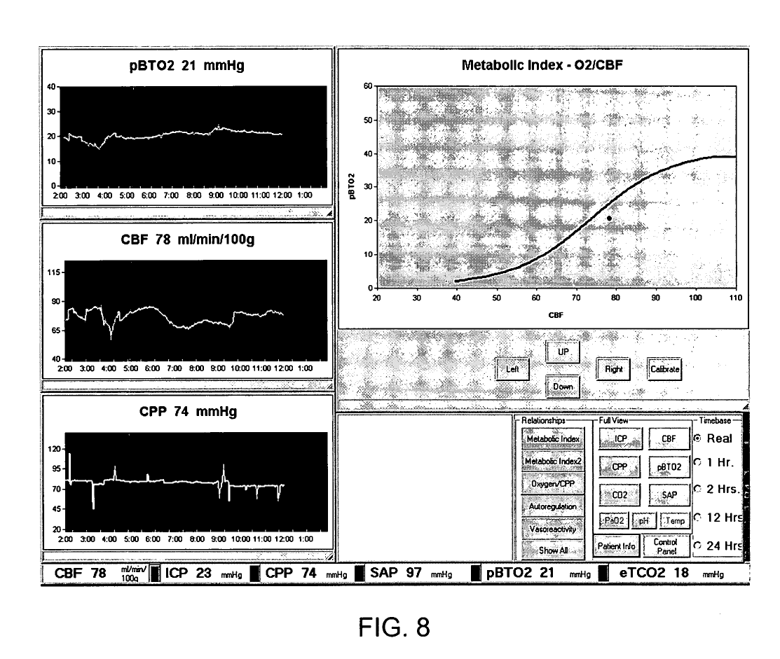

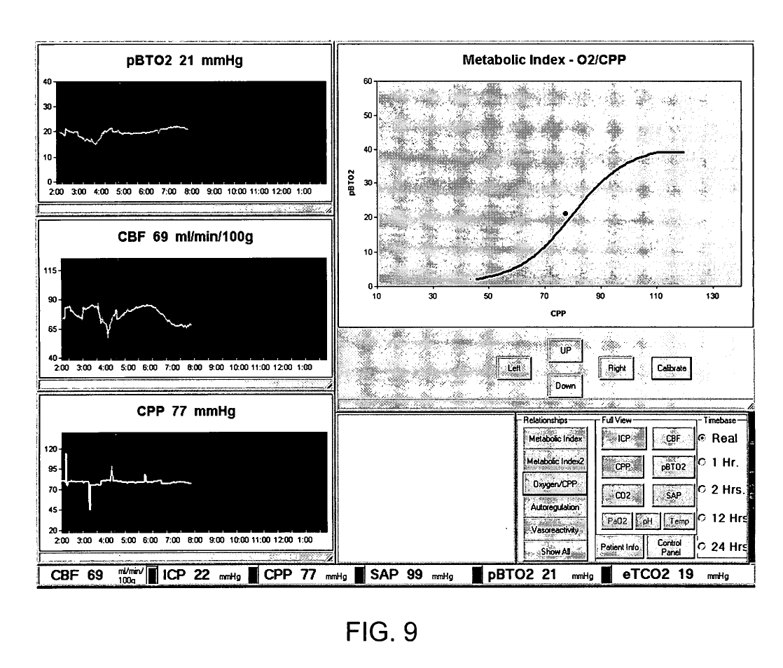

Referring to FIGS. 5-9, a user interface useable in connection with the illustrative system of FIG. 1 will now be described. This user interface can present single-variable views (see FIGS. 6-9) as well as a customizable multi-variable view (see FIG. 5). In the multi-variable view, two or more nominal relationship graphs 50, 60, 70 for derived variables are presented in connection with time plots showing values for more basic measurements that they are derived from 52, 54; 62, 64; 72. In this embodiment, the multi-variable view is accessed from a “show all” button in a main control panel 80.

The single-variable views can also be accessed from buttons in the main control panel. The autoregulation button, for example, allows a user to reach an autoregulation view (see FIG. 6). This view includes a graph of the nominal relationship 90 for the autoregulation derived variable as well as time plots for SAP, CBF, and eTCO2 92, 94, 96. A set of nominal relationship manipulation controls 84 allow the user to adjust or calibrate the autoregulation relationship. The relationship manipulation controls include an “up” button that shifts the autoregulation curve up, a “down” button that shifts the autoregulation curve down, a “left” button that shifts the autoregulation curve left, and a “right” button that shifts the autoregulation curve down.

Both types of views include an alphanumeric display bar 82 that presents a series of numerical values and corresponding units for different ones of the tracked variables. Proximate each of the variables is an indicator whose color indicates patient status for that variable. For example, green, yellow, and red can correspond to nominal, transitional, and exceptional, respectively.

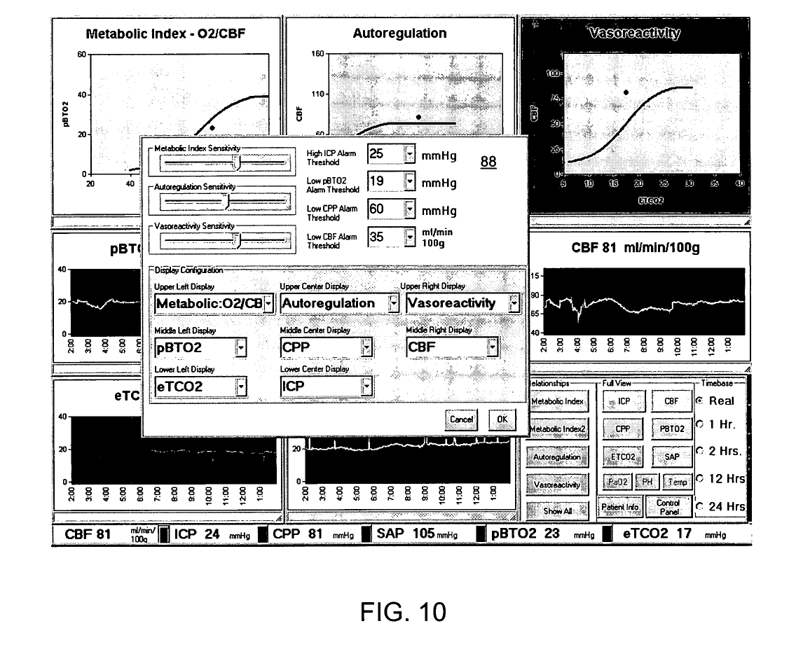

Referring to FIG. 10, more detailed customizations can be performed by accessing an auxiliary control panel 88 from the main control panel. This control panel allows sensitivities and alarms to be set for different derived variables. It also allows the user to select how the different parts of the multi-variable view and display bar will be presented. A timebase control allows the user to adjust the scale of displayed graphs.

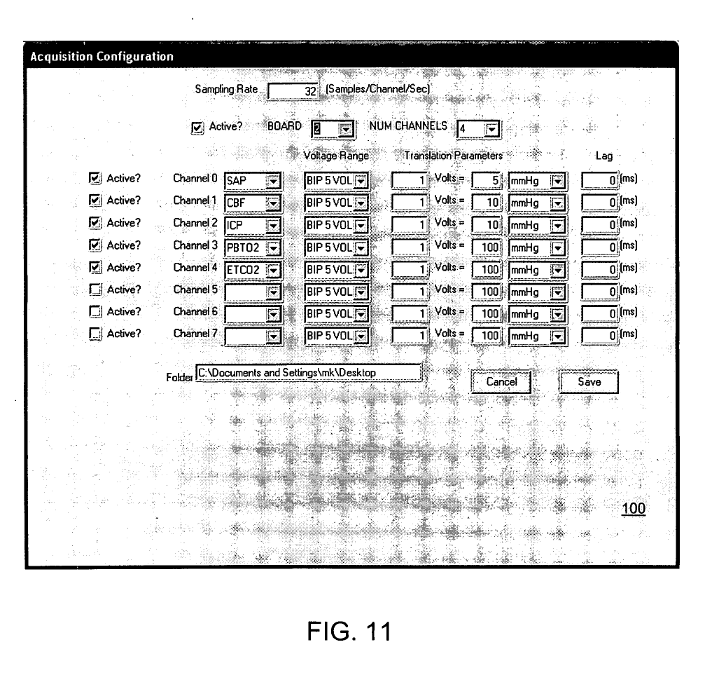

Referring to FIG. 11, the system can also provide an acquisition configuration dialog 100 that provides acquisition functionality that allows the user to set up the different measurement channels handled by the instrument. These channels can be activated or deactivated, and their input voltage ranges can be set. Translation parameters and lag values can also be provided so that different instruments work together as seamlessly as possible.

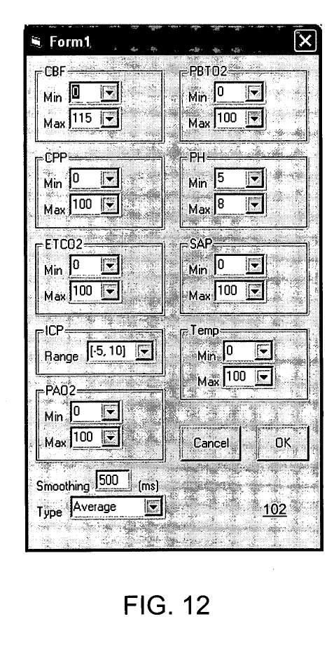

Referring to FIG. 12, the system can also provide a parameter range dialog 102. This dialog allows the user to set minimum and maximum values for parameters shown in the graphs. It also allows the user to specify a smoothing interval and a smoothing method. In this embodiment, the acquisition configuration dialog and the parameter range dialog can be reached through controls in the auxiliary control panel (not shown).

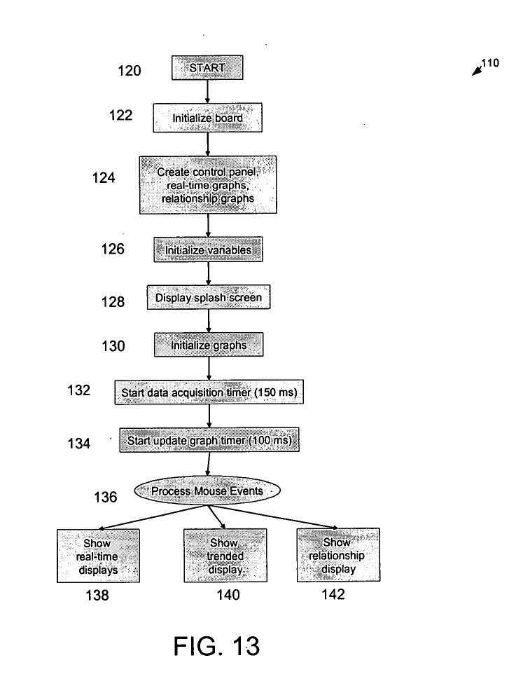

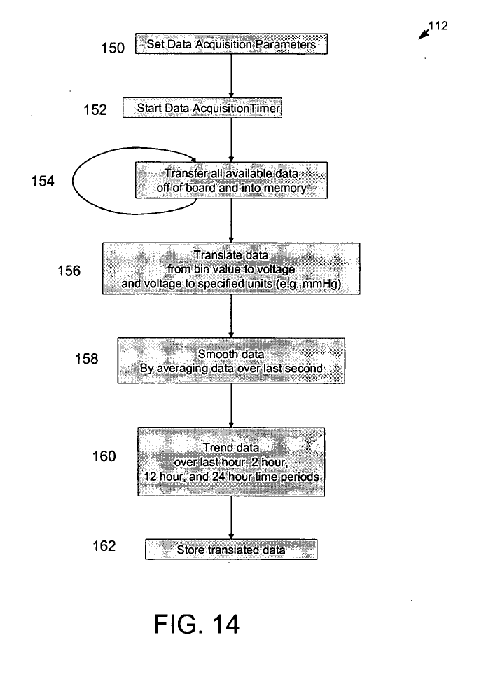

In operation, referring to FIG. 13, the system begins by performing a startup routine 110 in which the capture device (in this case a capture board) is first initialized (step 120). The control panel, real-time graphs, and relationship graphs are then created (step 122). Variables are then initialized, a splash screen is displayed, and graphs are initialized (steps 124, 126, 128, 130). A data acquisition timer is started (step 132) and an update graph timer is started (step 134). The system then begins processing mouse events to show real-time displays, trended displays, and/or relationship displays (steps 136, 138, 140, 142). 00661 Referring to FIG. 14, a separate acquisition timer routine 112 begins by setting acquisition parameters and starting a data acquisition timer (steps 150, 152). Data are then continuously transferred into memory until the data are translated from binary values to voltage and from voltage to specific units (steps 154, 156). The data can be smoothed, such as by averaging it over the last second (step 158). Data are then trended over last hour, two-hour, 12-hour, and 24-hour time periods, and stored (steps 160, 162).

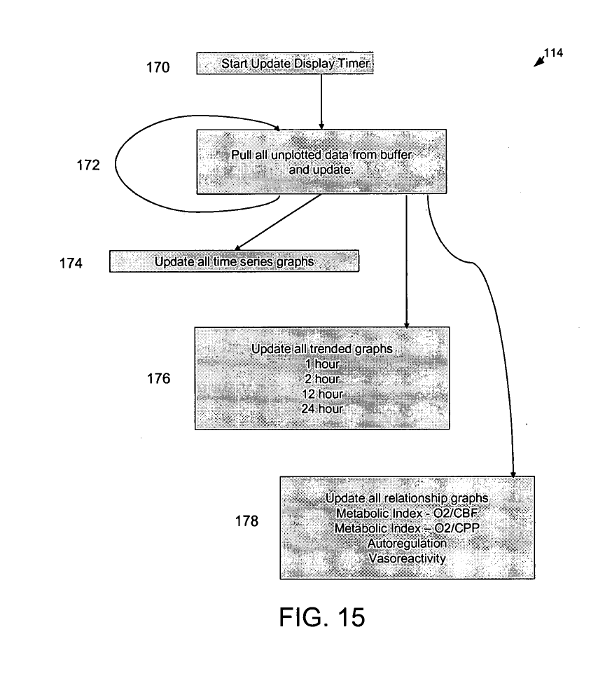

Referring to FIG. 15, a separate timer update routine 114 begins by setting an update display timer, and all unplotted data are pulled from the buffer and updated (steps 170, 172). The time series graphs are then updated, the trended graphs are updated, and the relationship graphs are updated (steps 174, 176, 178).

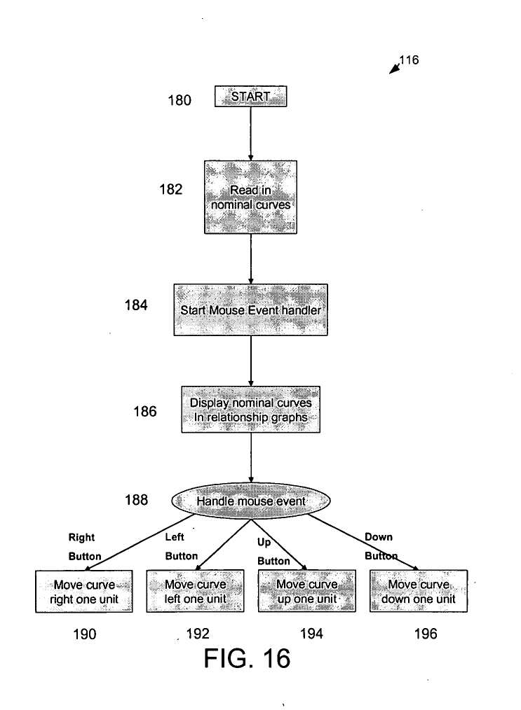

Referring to FIG. 16, a nominal curve calibration routine 116 begins by reading in the nominal curves, starting the mouse handler, and displaying the nominal curves in relationship graphs (steps 180, 182, 184, 186). The nominal curve calibration routine can then adjust the graphs in response to mouse events at the right, left, up, and down buttons on the main control panel (steps 188, 190, 192, 194, 196).

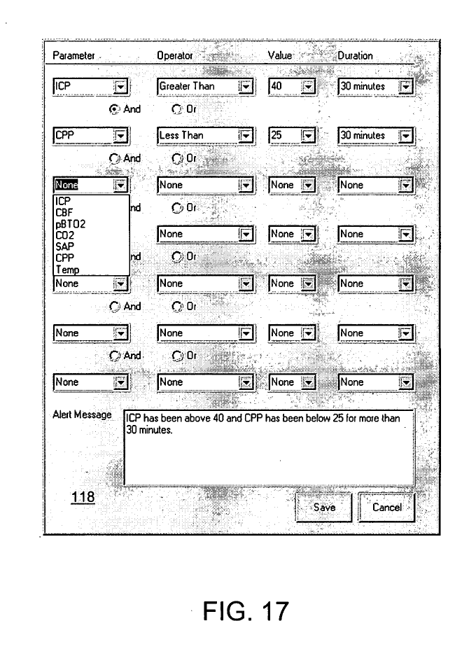

Referring to FIG. 17, the system can also provide a rule definition dialog 118. This dialog allows the user to define sets of rules that allow alerts or other types of events to take place in particular circumstances. These rules can operate on one or more of the basic or derived variables available in the system. This interface allows physicians, consultants, hospital committees, or other entities to define potentially complex sets of conditional events based on their needs and experience. And while the interface shown uses drop-down boxes to specify rules, they may also be specified in other ways, such as with other types of mouse-based or event driven controls, through the use of a dedicated text-based authoring tool, or by uploading rules prepared in a word processor.

In one embodiment, the system is implemented using off-the-shelf, standalone monitoring instruments connected to different inputs of an off-the-shelf data acquisition board, such as the Advantech PCI-1713 board, in a Windows®-based personal computer equipped with the Visual Basic programming environment. Of course, other structures, tools, and methodologies can be used to implement systems according to the invention, including various combinations of dedicated hardware and/or special-purpose software running on general-purpose hardware. In addition, the various elements and steps described can be reorganized, divided, and combined in different ways without departing from the scope and spirit of the invention. It should also be noted that not all of the items presented in the discussion of the illustrative embodiment will be required for all implementations of the invention.

This invention has now been described in connection with a number of specific embodiments thereof. However, numerous modifications which are contemplated as falling within the scope of this invention should now be apparent to those skilled in the art. For example, the clinical data can be output in other forms, such as auditory alarms, textual values, or simple indicator lights. And while the illustrative system described receives input from separate stand-alone instruments, it could also be part of an integrated instrument that incorporates all of the instrumentation necessary to calculate the derived variables. It is therefore intended that the scope of this invention be limited only by the scope of the claims appended hereto. In addition, the order of presentation of the claims should not be construed to limit the scope of any particular term in the claims.

1. A patient neuromonitoring system comprising:
a capture device having a first input responsive to a first basic measure of patient function, and a second input responsive to a second basic measure of patient function for the same patient, and
a process responsive to the capture device and operative to calculate at least one derived neurophysiological variable for the patient by combining the first and second basic measures of patient function, and to detect when the patient's state is different from a nominal state for the derived variable.

2. The apparatus of claim 1 wherein the process derives a measure of autoregulation from the first and second basic measures of patient function.

3. The apparatus of claim 2 wherein the capture device is responsive to cerebral blood flow measurements and arterial blood pressure measurements.

4. The apparatus of claim 2 wherein the capture device is responsive to cerebral perfusion pressure measurements and cerebral blood flow measurements.

5. The apparatus of claim 1 wherein the process derives a measure of carbon dioxide reactivity from the first and second basic measures of patient function.

6. The apparatus of claim 5 wherein the process derives a measure of carbon dioxide reactivity by comparing changes in the partial pressure of carbon dioxide and cerebral blood flow.

7. The apparatus of claim 1 wherein the process derives a measure of decoupling of cerebral blood flow and partial pressure of tissue oxygen from the first and second basic measures of patient function.

8. The apparatus of claim 7 wherein the process derives a measure of decoupling of cerebral blood flow by continually calculating measures indicating whether a relationship between cerebral blood flow increases and tissue oxygen increases is intact.

9. The apparatus of claim 1 wherein the process derives a measure of cerebral metabolic rate of oxygen consumption from the first and second basic measures of patient function.

10. The apparatus of claim 9 wherein the process derives a measure of cerebral metabolic rate of oxygen consumption from measures of cerebral blood flow, partial pressure of tissue oxygen, partial pressure of tissue carbon dioxide, intracranial pressure, and arterial blood pressure.

11. The apparatus of claim 1 further including means for providing the at least one derived variable to a clinician.

12. The apparatus of claim 11 wherein the means for providing the derived variables include a display operative to simultaneously display a plurality of the derived variable on a graph.

13. The apparatus of claim 11 wherein the means for providing the derived variables include a display operative to display the at least one derived variable on a separate graph.

14. The apparatus of claim 1 further including a database responsive to the capture device and wherein the capture device is operative to persistently store data from the first and second inputs.

15. The apparatus of claim 1 wherein the capture device is further operative to transform the output of various neurosurgical monitoring devices into a form that is suitable for storing in a database.

16. The apparatus of claim 1 wherein the first and second inputs of the capture device are responsive to the output of one or more neurosurgical monitoring devices.

17. The apparatus of claim 1 wherein the capture device is operative to receive analog or digital signals.

18. The apparatus of claim 1 wherein the process is operative to detect anomalous conditions from a plurality of rules.

19. The apparatus of claim 18 wherein the process further includes a rule definition interface to define the rules.

20. The apparatus of claim 1 wherein the capture device and the process are operative on a continuous basis to obtain continuous values for the derived variable.

21. The apparatus of claim 1 wherein the capture device and the process are operative on a real-time basis to obtain real-time values for the derived variable.

22. The apparatus of claim 1 wherein the nominal state is derived from measurements made during a baseline period.

23. The apparatus of claim 1 wherein the nominal state is derived from a clinical understanding of the derived variable.

24. A patient neuromonitoring method, comprising:
receiving at least two different measures of patient condition from a patient,
calculating at least one derived neurophysiological variable for the patient based on the two different measures of patient condition in a continuous manner, and
detecting when the patient's state is different from a nominal state for the derived variable.

25. The method of claim 24 further including the step of outputting the derived neurophysiological variable to a clinician.

26. The method of claim 24 further including the step of displaying the derived neurophysiological variable to a clinician.

27. The method of claim 24 wherein the step of detecting is responsive to a set of rules.

28. The method of claim 24 wherein the steps of receiving, calculating, and detecting operate on a continuous basis.

29. The method of claim 24 wherein the steps of receiving, calculating, and detecting operate on a real-time basis.

30. A patient neuromonitoring system, comprising:
means for receiving at least two different measures of patient condition from a patient,
means for calculating at least one derived neurophysiological variable for the patient based on the two different measures of patient condition in a continuous manner, and
means for detecting when the patient's state is different from a nominal state for the derived variable.

31. A patient neuromonitoring system comprising:
a capture device having a first input responsive to a first basic measure of patient function, and a second input responsive to a second basic measure of patient function for the same patient, and being operative to persistently store data from the first and second inputs, and
a process responsive to the capture device and operative to calculate at least one derived neurophysiological variable for the patient in a continuous manner by combining the first and second basic measures of patient function.

32. The apparatus of claim 31 further including a database responsive to the capture device and wherein the capture device is operative to persistently store data from the first and second inputs in the database.

33. The apparatus of claim 31 further including a database responsive to the process and wherein the process is operative to persistently store values for the derived neurophysiological variable in the database.

34. A patient neuromonitoring method, comprising:
receiving at least two different measures of patient condition from a patient,
calculating at least one derived neurophysiological variable for the patient based on the two different measures of patient condition in a continuous manner, and
persistently storing at least one of the two different measures of patient condition and the derived neurophysiological variable.

35. A patient neuromonitoring system comprising:
a capture device having a first input responsive to a first basic measure of patient function, a second input responsive to a second basic measure of patient function for the same patient, and a third input responsive to a third measure of patient function for the same patient, and
a process responsive to the capture device and operative to calculate at least one derived neurophysiological variable for the patient by combining a subset of the first, second, and third basic measures of patient function.

36. The apparatus of claim 35 wherein the process is responsive to the capture device and operative to calculate a plurality of different derived neurophysiological variables for the patient by combining subsets of the basic measures of patient function.

37. A patient neuromonitoring method, comprising:
receiving different measures of patient condition from a patient,
calculating a first derived neurophysiological variable for the patient based on a first subset of the different measures of patient condition in a continuous manner, and
calculating a second derived neurophysiological variable for the patient based on a second subset of the different measures of patient condition in a continuous manner, wherein the first and second subsets are different.
